(19) United States
(12) Patent Application Publication
(10) Pub. No.: US 2005/0037052 A1
(43) Pub. Date: Feb. 17, 2005

(54) Stent coating with gradient porosity

(75) Inventors: Kishore Udipi, Santa Rosa, CA (US); Peiwen Cheng, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 10/917,849

(22) Filed: Aug. 12, 2004

Related U.S. Application Data
(60) Provisional application No. 60/495,206, filed on Aug. 13, 2003.

(51) Int. Cl. A61F 2/00

(52) U.S. Cl. 424/426

(57) ABSTRACT

Biocompatible coatings for medical devices are disclosed. Specifically, polymer coatings designed to control the release of drugs from medical devices in vivo are disclosed wherein the porosity of the polymer coatings is varied to control elute rate profiles. Also disclosed are vascular stents and stent grafts with controlled release coatings and related methods for making these coatings.

### RELATED APPLICATIONS

This application claims priority to U.S. provisional patent application Ser. No. 60/495,206 filed Aug. 13, 2003.

### FIELD OF THE INVENTION

The present invention relates generally to biocompatible coatings for medical devices. More specifically, the present invention relates to polymer coatings designed to control the release of drugs from a medical device. The present invention provides vascular implants with controlled release coatings containing drugs and related methods for making these coating. Additionally the present invention provides methods for controlling release of drugs by coating medical devices with successive layers of polymer coatings of different porosities.

### BACKGROUND OF THE INVENTION

The drug-coated stent is a very active research and development area in stent manufacture. In practice, a common solvent or pair of solvents is used to dissolve a drug and polymer (including copolymers or polymer blends). Then the drug/polymer solution is applied to the stents. After application, the drug/polymer reservoir (film) is formed on the stent surface. In this process, for each formulation, the drug/polymer ratio and polymer content are fixed. When the drug-coated stent is deployed in a vessel in the body, the drug release is based on a diffusion mechanism.

The drug diffusion is controlled by many factors, such as the molecular size of the drug, its crystallinity and hydrophil/lipophil balance, the morphology of the coating, and the glass transition point (Tg) of the polymer matrix. However, a common releasing profile is observed most of the time. In this common releasing profile a large amount of drug is released first (burst release) followed by a slow and gradual release leading to a plateauing effect. This occurs due to the resistance offered by the polymer film to the transport of drug to the surface.

There remains a need in the art for compositions and methods which allow medical devices to be easily and efficiently coated with a wide variety of pharmaceutical agents, and that further provides controlled or sustained release of the pharmaceutical agents into the local area surrounding the site of medical intervention. Additionally, there remains a need in the art for a method which will expedite or speed up the transport of the drug from the inner layers, next to the stent surface, to the outer edge of the polymer film.

### SUMMARY OF THE INVENTION

The present invention provides a method for expediting the transport of drug from the inner layers of the polymer film (which is next to the stent surface) to the outer edge of the polymer film. More specifically, the present invention provides a method for overcoming the plateauing effect and maintaining a steady release of the drug by introducing porosity in the inner layers of the polymer film.

In summary, a drug-polymer coated stent having a steady drug release is prepared by preparing a first drug polymer solution. The first drug polymer solution is deposited onto the surface of a medical device, such as a stent, thereby creating a first coated layer which has a first porosity value. Additionally, a second drug polymer solution is prepared. The second drug polymer solution is deposited onto the first coated layer thereby creating a second coated layer which has a second porosity value. This second porosity value is less than the first porosity value. The result is a drug-polymer coated stent having a steady drug release.

In other embodiments of the present invention, multiple coating layers are applied (e.g.: a first, second, third, fourth coating and so on) each coating having progressively smaller porosity values the farther away from the device surface.

A non-solvent may be added to the first drug polymer solution. Further, a non solvent may be added to the second drug polymer solution. Additionally, one or more additional drug polymer solutions may be prepared. Any additional prepared drug solutions may be deposited onto the drug-polymer coated stent, thereby creating one or more additional layer. Any additional layers are deposited onto the drug-polymer coated stent such that each successive drug polymer solution applied has a lower porosity value.

If, in the preparation of the first drug polymer solution, a non-solvent is added, the created mixture may be of about 95% CHCl₃ and about 5% CH₃OH. Alternatively, if in the preparation of the first drug polymer solution a non-solvent is added, the created mixture may be of about 70% CHCl₃ and about 30% CH₃OH. In a different embodiment of the invention, if no non-solvent is added in the preparation of the first drug polymer solution, then the created mixture may be of about 100% CHCl₃.

In another embodiment of the invention the drug polymer coating is comprised of varying porosity phases. The first drug polymer layer having a first porosity value may be made from a first drug polymer solution and the second drug polymer layer having a second porosity value may be made from a second drug polymer solution.

An additional embodiment of the invention provides a method for preparing a stent. In summary, the first step is providing a stent having an outer surface. The next step is depositing a first drug-polymer solution adjacent to the outer surface of the stent thereby creating a first layer having a first inner surface and a first outer surface, the first inner surface of the first layer being directly adjacent to the outer surface of the stent. The following step is depositing a second drug-polymer solution adjacent to the outer surface of the first layer thereby creating a second layer having a second inner surface and a second outer surface, the second inner surface of the second layer being directly adjacent to the first outer surface of the first layer.

### BRIEF DESCRIPTION OF THE DRAWINGS

FIG. 1 is a graphical illustration representing a releasing profile of a drug/polymer matrix made in accordance with the teachings of the present invention.

FIG. 2 depicts a scanning electron micrograph (SEM) of an expanded stent segment having a coating made in accordance with the teachings of the present invention.

### DEFINITION OF TERMS

Prior to setting forth the invention, it may be helpful to an understanding thereof to set forth definitions of certain terms that will be used hereinafter:

Animal: As used herein “animal” shall include mammals, fish, reptiles and birds. Mammals include, but are not limited to, primates, including humans, dogs, cats, goats, sheep, rabbits, pigs, horses and cows.

Biocompatible: As used herein “biocompatible” shall mean any material that does not cause injury or death to the animal or induce an adverse reaction in an animal when placed in intimate contact with the animal's tissues. Adverse reactions include inflammation, infection, fibrotic tissue formation, cell death, or thrombosis.

Cap coat: As used herein “cap coat” refers to the outermost coating layer applied over another coating.

Controlled release: As used herein “controlled release” refers to the release of a bioactive compound from a medical device surface at a predetermined rate. Controlled release implies that the bioactive compound does not come off the medical device surface sporadically in an unpredictable fashion and does not “burst” off of the device upon contact with a biological environment (also referred to herein a first order kinetics) unless specifically intended to do so. However, the term “controlled release” as used herein does not preclude a “burst phenomenon” associated with deployment. In some embodiments of the present invention an initial burst of drug may be desirable followed by a more gradual release thereafter. The release rate may be steady state (commonly referred to as “timed release” or zero-order kinetics), that is the drug is released in even amounts over a predetermined time (with or without an initial burst phase) or may be a gradient release. A gradient release implies that the concentration of drug released from the device surface changes over time.

Compatible: As used herein “compatible” refers to a composition possess the optimum, or near optimum combination of physical, chemical, biological and drug release kinetic properties suitable for a controlled release coating made in accordance with the teachings of the present invention. Physical characteristics include durability and elasticity/ductility, chemical characteristics include solubility and/or miscibility and biological characteristics include bibcompatibility. The drug release kinetic should be either near zero-order or a combination of first and zero-order kinetics.

Drug(s): As used herein “drug” shall include any bioactive agent having a therapeutic effect in an animal. Exemplary, non limiting examples include anti-proliferatives including, but not limited to, macrolide antibiotics including FKBP 12 binding compounds, estrogens, chaperone inhibitors, protease inhibitors, protein-tyrosine kinase inhibitors, peroxisome proliferator-activated receptor gamma (PPAR gamma) ligands, hypothemycin, nitric oxide, bisphosphonates, anti-proliferatives, paclitaxel, epidermal growth factor inhibitors, antibodies, proteasome inhibitors, antibiotics, anti-sense nucleotides, transforming nucleic acids and matrix metalloproteinase inhibitors.

Glass transition point: As used herein “glass transition point” or “Tg” is the temperature at which an amorphous polymer becomes hard and brittle like glass. At temperatures above its Tg a polymer is elastic or rubbery; at temperatures below its Tg the polymer is hard and brittle like glass. Tg may be used as a predictive value for elasticity/ductility.

Non-solvent: As used herein “non-solvent” refers to a solvent which causes a polymer to precipitate out of solution. A non-solvent can be of opposite polarity to the solvent or can differ in its solubility profile regarding the polymer.

Treatment site: As used herein “treatment site” shall mean a vascular occlusion or aneurysm site.

### DETAILED DESCRIPTION OF THE INVENTION

The present invention is directed at engineering polymers that provide optimized drug-eluting medical devices coatings. Specifically, polymers made in accordance with teachings of the present invention provide durable biocompatible coatings for medical devices intended for use in hemodynamic environments. In one embodiment of the present invention vascular stents are coated using the polymer compositions of the present invention. In another embodiment of the present invention stent grafts are coated using the polymer compositions of the present invention. Vascular stents and stent grafts are chosen for exemplary purposes only. Those skilled in the art of material science and medical devices will realize that the polymer compositions of the present invention are useful in coating a large range of medical devices. Therefore, the use of vascular stents and stent grafts as exemplary embodiments is not intended as a limitation.

Vascular stents and stent grafts (referred to hereinafter collectively as “stents”) present a particularly unique challenge for the medical device coating scientist. Stents must be flexible, expandable, biocompatible and physically stable. Stents are used to relieve the symptoms associated with coronary artery disease caused by occlusion in one or more coronary artery or aneurysms. Occluded coronary arteries result in diminished blood flow to heart muscles causing ischemia induced angina and in severe cases myocardial infarcts and death. Stents are generally deployed using catheters having the stent attached to an inflatable balloon at the catheter's distal end. The catheter is inserted into an artery and guided to the deployment site. In many cases the catheter is inserted into the femoral artery or of the leg or carotid artery and the stent is deployed deep within the coronary vasculature at an occlusion site.

Vulnerable plaque stabilization is another application for coated drug-eluting vascular stents. Vulnerable plaque is composed of a thin fibrous cap covering a liquid-like core composed of an atheromatous gruel. The exact composition of mature atherosclerotic plaques varies considerably and the factors that effect an atherosclerotic plaque's make-up are poorly understood. However, the fibrous cap associated with many atherosclerotic plaques is formed from a connective tissue matrix of smooth muscle cells, types I and III collagen and a single layer of endothelial cells. The atheromatous gruel is composed of blood-borne lipoproteins trapped in the sub-endothelial extracellular space and the breakdown of tissue macrophages filled with low density lipids (LDL) scavenged from the circulating blood. (G. Pasterkamp and E. Falk. 2000. Atherosclerotic Plaque Rupture: An Overview. J. Clin. Basic Cardiol. 3:81-86). The ratio of fibrous cap material to atheromatous gruel determines plaque stability and type. When atherosclerotic plaque is prone to rupture due to instability it is referred to a “vulnerable” plaque. Upon rupture the atheromatous gruel is released into the blood stream and induces a massive thrombogenic response leading to sudden coronary death. Recently, it has been postulated that vulnerable plaque can be stabilized by stenting the plaque. Moreover, vascular stents having a drug-releasing coating composed of matrix metalloproteinase inhibitor (such as, but not limited to, tetracycline-class antibiotics) dispersed in, or coated with (or both) a polymer may further stabilize the plaque and eventually lead to complete healing.

Treatment of aneurysms is another application for drug-eluting stents. An aneurysm is a bulging or ballooning of a blood vessel usually caused by atherosclerosis, aneurysms occur most often in the abdominal portion of the aorta. At least 15,000 Americans die each year from ruptured abdominal aneurysms. Back and abdominal pain, both symptoms of an abdominal aortic aneurysm, often do not appear until the aneurysm is about to rupture, a condition that is usually fatal. Stent grafting has recently emerged as an alternative to the standard invasive surgery. A vascular graft containing a stent (stent graft) is placed within the artery at the site of the aneurysm and acts as a barrier between the blood and the weakened wall of the artery, thereby decreasing the pressure on artery. The less invasive approach of stent-grafting aneurysms decreases the morbidity seen with conventional aneurysm repair. Additionally, patients whose multiple medical comorbidities make them excessively high risk for conventional aneurysm repair are candidates for stent-grafting. Stent grafting has also emerged as a new treatment for a related condition, acute blunt aortic injury, where trauma causes damage to the artery.

Once positioned at the treatment site the stent or graft is deployed.

Generally, stents are deployed using balloon catheters. The balloon expands the sent gently compressing it against the arterial lumen clearing the vascular occlusion or stabilizing the plaque. The catheter is then removed and the stent remains in place permanently. Most patients return to a normal life following a suitable recovery period and have no reoccurrence of the arterial disease associated with the stented deployment. However, in some cases the arterial wall's initma is damaged either by the disease process itself or as the result of stent deployment. This injury initiates a complex biological response culminating in vascular smooth muscle cell hyperproliferation and occlusion, or restenosis, at the stent site.

Recently significant efforts have been devoted to preventing restenosis. Several techniques including brachytherapy, excimer laser, and pharmacological interventions have been developed. The least invasive and most promising treatment modality is the pharmacological approach. A preferred pharmacological approach involves the site specific delivery of cytostatic or cytotoxic drugs directly to the stent deployment area. Site specific delivery is preferred over systemic delivery for several reasons. First, many cytostatic and cytotoxic drugs are highly toxic and cannot be administered systemically at concentrations needed to prevent restenosis. Moreover, the systemic administration of drugs can have unintended side effects at body locations remote from the treatment site. Additionally, many drugs are either not sufficiently soluble, or too quickly cleared from the blood stream to effectively prevent restenosis. Therefore, administration of anti-restenotic compounds directly to the treatment area is preferred.

Several techniques and corresponding devices have been developed to deploy drugs including weeping balloon and injection catheters. Weeping balloon catheters are used to slowly apply an anti-restenotic composition under pressure through fine pores in an inflatable segment at or near the catheter's distal end. The inflatable segment can be the same used to deploy the stent or separate segment. Injection catheters administer the anti-restenotic composition by either emitting a pressurized fluid jet, or by directly piercing the artery wall with one or more needle-like appendage. Recently, needle catheters have been developed to inject drugs into an artery's adventitia. However, administration of drugs using weeping and injection catheters to prevent restenosis remains experimental and largely unsuccessful. Direct drug administration has several disadvantages. When drugs are administered directly to the arterial lumen using a weeping catheter, the blood flow quickly flushes the anti-restenotic composition down stream and away from the treatment site. Drug compositions injected into the lumen wall or adventitia may rapidly diffuse into the surrounding tissue. Consequently, drug compositions may not be present at the treatment site in sufficient concentrations to prevent restenosis. As a result of these and other disadvantages associated with catheter-based local drug delivery, investigators continue to seek improved methods for the localized delivery of anti-restenotic compositions.

The most successful method for localized drug composition delivery developed to date is the drug-eluting stent. Many drug-eluting stent embodiments have been developed and tested. However, significant advances are still necessary in order to provide safe and highly effective drug delivery stents. One of the major challenges is controlling the drug delivery rate. Factors affecting drug delivery include coating composition, coating configurations, polymer swellability and coating thickness. When the medical device of the present invention is used in the vasculature, the coating dimensions are generally measured in micrometers (um). Coatings consistent with the teaching of the present invention may be a thin as 1 um or a thick as 1000 um. There are at least two distinct coating configurations within the scope of the present invention. In one embodiment of the present invention the drug-containing coating is applied directly to the device surface or onto a polymer primer coat such a parylene or a parylene derivative. Depending on the solubility rate and profile desired, the drug is either entirely soluble within the polymer matrix, or evenly dispersed throughout. The drug concentration present in the polymer matrix ranges from 0.1% by weight to 80% by weight. In either event, it is most desirable to have as homogenous a coating composition as possible. This particular configuration is commonly referred to as a drug-polymer matrix.

In another embodiment of the present invention, a drug-free polymer barrier, or cap, coat is applied over the drug-containing coating. The drug-containing coating serves as a drug reservoir. Generally, the concentration of drug present in the reservoir ranges from about 0.1% by weight to as much as 100%. The barrier coating participates in controlling drug release rates in at least three ways. In one embodiment the barrier coat has a solubility constant different from the underlying drug-containing coating. In this embodiment, the drug's diffusivity through the barrier coat is regulated as a function of the barrier coating's solubility factors. The more miscible the drug is in the barrier coat, the quicker it will elute form the device surface and visa versa. This coating configuration is commonly referred to as a reservoir coating.

In another embodiment the barrier coat comprises a porous network where the coating acts as a molecular sieve. The larger the pores relative to the size of the drug, the faster the drug will elute. Moreover, intramolecular interactions will also determine the elution rates. Finally, returning to coating thickness, while thickness is generally a minor factor in determining overall drug-release rates and profile, it is never-the-less an additional factor that can be used to tune the coatings. Basically, if all other physical and chemical factors remain unchanged, the rate at which a given drug diffuses through a given coating is inversely proportional to the coating thickness. That is, increasing the coating thickness decreases the elution rate and visa versa.

The controlled release coatings of the present invention can be applied to medical device surfaces, either primed or bare, in any manner known to those skilled in the art. Applications methods compatible with the present invention include, but are not limited to, spraying, dipping, brushing, vacuum-deposition, and others. Moreover, the controlled release coatings of the present invention may be used with a cap coat. For example, and not intended as a limitation: a metal stent has a parylene primer coat applied to its bare metal surface. Over the primer coat a drug-releasing polymer coating or blend of polymers is applied. Over the drug-containing coating a polymer cap coat is applied. The cap coat may optionally serve as a diffusion barrier to further control the drug release, or provide a separate drug. The cap coat may be merely a biocompatible polymer applied to the surface of the stent to protect the stent and have no effect on elusion rates.

Drug-eluting polymer coatings for medical devices are becoming increasingly more common. Furthermore, the number of possible polymer-drug combinations is increasing exponentially. Therefore, there is need for reproducible methods of designing drug-polymer compositions such that drug-elution rates/profiles, biocompatibility and structural integrity are compatibilized resulting in optimal coating systems tailored for specific therapeutic functions. The present invention provides both exemplary optimal coating systems and related methods for their reproducible design.

The present invention describes method(s) to prepare stent coatings with gradient porosity to modulate release of incorporated drug from the coatings. More particularly, the present invention relates to a method for expediting the transport of the incorporated drug from the inner coatings to the outer edge of the outer layer.

The porosity gradient in the coating is attained by phase separation. Addition of a non-solvent to the polymer solution leads to phase separation. The higher the amount of non-solvent, the higher the degree of phase separation and the higher the porosity in the film. The coat next to the stent surface is formulated with the highest amount of non-solvent to exhibit the most porosity. Successive coats of drug-polymer solutions are formulated with decreasing amounts of non-solvent which will provide a coating system with progressively lower porosity.

The examples are meant to illustrate one or more embodiments of the invention and are not meant to limit the invention to that which is described below.

### EXAMPLE 1

### Preparation of Solution 1, a 1% Drug/polymer Solution (95% CHCl₃, 5% CH₃OH)

In one embodiment of the invention, a 1% drug/polymer solution (95% CHCl₃, 5% CH₃OH) is prepared. This solution may be prepared by the following steps. First, combine 0.0187 g of rapamycin and 0.0224 g of poly(butyl methacrylate-co-methyl methacrylate) Aldrich cat # 47403-7 into a container such as a glass vial. Next, add 0.0337 g of poly(ethylene-co-vinyl acetate). (PEVA) to the same glass vial with rapamycin. Then, add 4.7 ml of chloroform and 0.5 ml of methanol to the glass vial. Finally, shake the vial until all materials have dissolved. For purposes of illustration only, this solution will be referred to as Solution 1.

### EXAMPLE 2

### Preparation of Solution 2, a 1% Drug/polymer Solution (70% CHCl₃, 30% CH₃OH)

One example of preparing a 1 % drug/polymer solution (70% CHCl₃, 30% CH₃OH) is illustrated in the following steps. First, weigh 0.1442 g of rapamycin in a glass bottle. Second, weigh 0.1730 g of poly(butyl methacrylate-comethyl methacrylate) Aldrich cat # 47403-7 in a weighing pan and transfer the weighed material into the same glass vial with rapamycin. Third, weigh 0.2576 g of PEVA in a weighing pan and transfer into the same glass vial with rapamycin. Next, add 26.7 ml of chloroform and 21.6 ml of methanol into the glass vial. Finally, shake the vial until all materials have dissolved. For purposes of illustration only, this solution will be referred to as Solution 2.

### EXAMPLE 3

### Preparation of Solution 3, a 1% Drug/polymer Solution (100% CHCl₃)

The following steps illustrate a method for preparing a 1% drug/polymer solution (100% CHCl₃). First, weigh 0.0454 g of rapamycin in a glass bottle. Second, weigh 0.0542 g of poly(butyl methacrylate-comethyl methacrylate) Aldrich cat # 47403-7 in a weighing pan and transfer it into the same glass vial with rapamycin. Third, weigh 0.0813 g of PEVA in a weighing pan and transfer it into the same glass vial with rapamycin. Fourth, add 12 ml of chloroform into the bottle. Finally, shake the bottle well until materials have dissolved. For purposes of illustration only, this solution will be referred to as Solution 3.

The following two examples illustrate the preparation of different coat stents using the solutions prepared in the above examples (Solution 1, Solution 2 and Solution 3).

### EXAMPLE 4

### Preparation of Coated Stent 1

In this first coated stent example, Solution 1 and Solution 3 from the above examples are used. To prepare Coated Stent 1, Solution 1 is sprayed onto a 9 mm stent. The target weight is 300 μg. After spraying the stent with Solution 1, the stent is preferably dried. Once the stent is dry, Solution 1 is sprayed onto the same stent. The target weight is 100 μg. Then the stent should be dried at room temperature overnight.

Finally, the dried stent is annealed at 45° C. for two hours.

### EXAMPLE 5

### Preparation of Coated Stent 2

In this second coated stent example, Solution 2 and Solution 3 illustrated in the above examples are used. To prepare Coated Stent 2, Solution 2 is sprayed onto a 9 mm stent. The target weight is 300 μg. The sprayed stent is then dried. After the stent had dried, Solution 3 is sprayed onto the same stent. The target weight is 100 μg. The stent then is dried at room temperature overnight. Once the stent had dried, the stent is annealed at 45° C. for two hours.

### EXAMPLE 6

### Releasing Profile

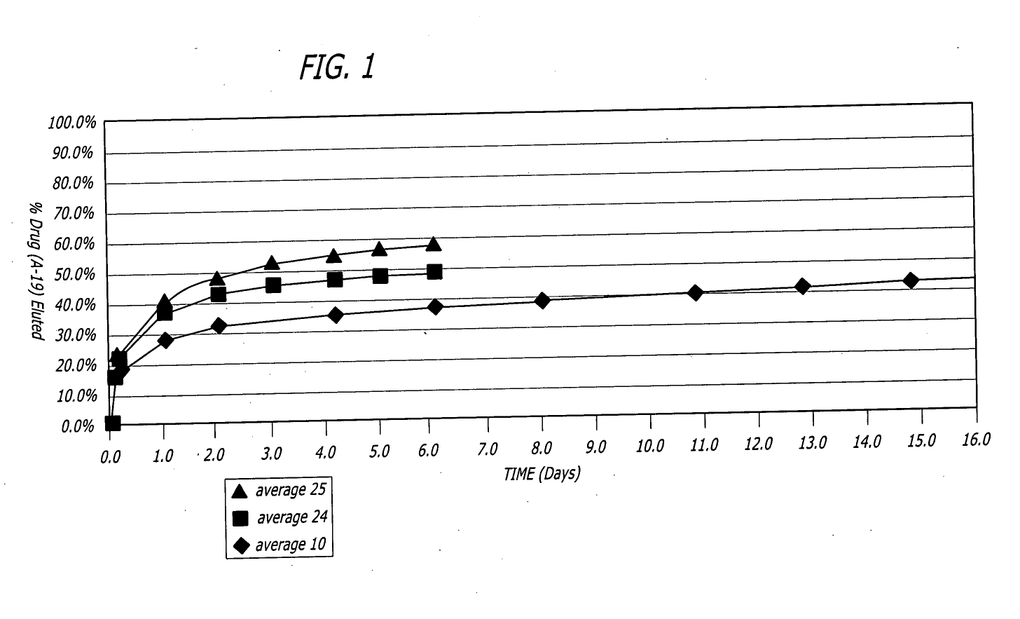

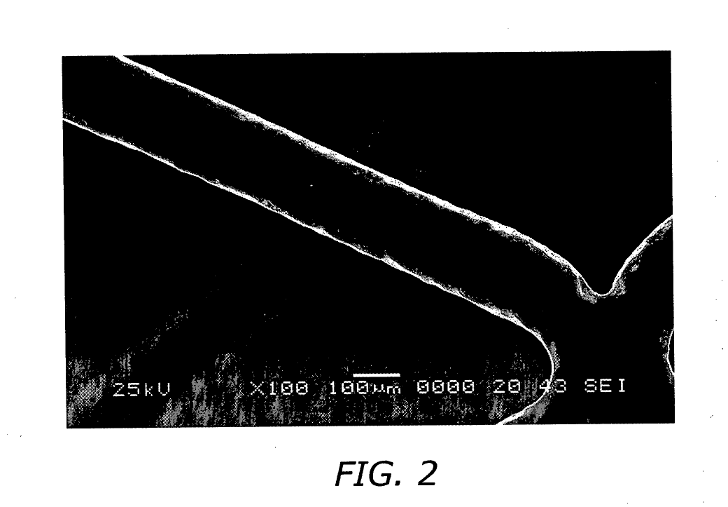

After preparing the above described coated stents, the elution of the drug was observed and recorded. From the resulting observed data, releasing profiles were created. FIG. 1 illustrates the amount of a drug eluted over a period of days using the three different drug/polymer solutions: #10, 100% CHCl₃; #24, 95% CHCl₃, 5% CH₃OH; #25, 70% CHCl₃, 30% CH₃OH. Additionally, FIG. 2 depicts a scanning electron micrograph (SEM) of an expanded stent segment having a coating made in accordance with the teachings of the present invention.

Those skilled in the art will further appreciate that the present invention may be embodied in other specific forms without departing from the spirit or central attributes thereof. In that the foregoing description of the present invention discloses only exemplary embodiments thereof, it is to be understood that other variations are contemplated as being within the scope of the present invention. Accordingly, the present invention is not limited in the particular embodiments which have been described in detail therein. Rather, reference should be made to the appended claims as indicative of the scope and content of the present invention.

1. An implantable medical device having a controlled release coating comprising a first drug-containing polymer layer having a first porosity value and a second drug-containing polymer layer having a second porosity value wherein said second porosity value is less than said first porosity value.

2. The controlled release coating according to claim 1 further comprising a third drug-containing polymer layer having a third porosity value wherein said third porosity value is less than said second porosity value.

3. The controlled coating according to claim 1 wherein said drug is selected from the group consisting of macrolide antibiotics, estrogens, chaperone inhibitors, protease inhibitors, protein-tyrosine kinase inhibitors, peroxisome proliferator-activated receptor gamma ligands, hypothemycin, nitric oxide, bisphosphonates, anti-proliferatives, paclitaxel, epidermal growth factor inhibitors, antibodies, proteasome inhibitors, antibiotics, anti-sense nucleotides, transforming nucleic acids and protease inhibitors.

4. The controlled coating according to claim 3 wherein said macrolide antibiotic is FKBP 12 binding compound.

5. The controlled release coating according to claim 1 wherein said medical device is a vascular stent or stent graft.

6. A vascular stent having a controlled release coating comprising a first FKBP 12 binding compound-containing polymer layer having a first porosity value disposed on the surface of said stent and a second FKBP 12 binding compound-containing polymer layer having a second porosity value disposed over said first layer wherein said first porosity value is greater than said second porosity value.

7. The vascular stent according to claim 6 further comprising a primer coat between said stent surface and said first FKBP 12 binding compound containing polymer layer.

8. The vascular stent according to either of claims 6 or 7 further comprising a polymer cap coat over said second FKBP 12 binding compound containing polymer layer.

9. The vascular stent according to claim 7 wherein in said primer coat is comprised of parylene.

10. The controlled release coating according to either of claims 1 or 6 wherein said first polymer layer comprises poly butyl methacrylate-co-methyl methacrylate and said second polymer layer comprises polyethylene vinyl acetate.

11. The controlled release coating according to claim 1 wherein said first drug-containing polymer comprises a first drug and said second drug-containing polymer comprises a second drug different from said first drug.

12. A method for treating a vascular disease in a mammal comprising placing a vascular stent or stent graft at a treatment site within a vessel wherein said vascular stent or stent graft has a controlled release coating comprising a first drug-containing polymer layer having a first porosity value and a second drug-containing polymer layer having a second porosity value wherein said second porosity value is less than said first porosity value.

13. The method for treating a vascular disease in a mammal according to claim 12 further comprising using a balloon catheter to place said stent or stent graft at said treatment site within said vessel.

14. The method for treating a vascular disease in a mammal according to claim 12 wherein said vascular disease is selected from the group consisting of restenosis, vulnerable plaque and aneurysms.

15. A method for preparing a controlled release coating for a medical device comprising:
depositing a first drug-polymer solution onto the surface of a medical device thereby creating a first coated layer with a first porosity value; and
depositing a second drug-polymer solution onto the first coated layer thereby creating a second coated layer with a second porosity value.

16. The method for preparing a controlled release coating according to claim 15 wherein the first porosity value is less than the second porosity value.

17. The method for preparing a controlled release coating according to claim 15 wherein the second porosity value is less than the first porosity value.

18. The method for preparing a controlled release coating according to claim 15 wherein the step of preparing the first drug polymer solution further comprises the step of adding a non-solvent to the first drug polymer solution.

19. The method for preparing a controlled release coating according to claim 15 wherein the step of preparing the second drug polymer solution further comprises the step of adding a non-solvent to the second drug polymer solution.

20. The method for preparing a controlled release coating according to claim 19 wherein the step of depositing the one or more than one additional drug polymer solution results in each successive drug polymer solution applied having a lower porosity value.

21. The method for preparing a controlled release coating according to claim 15 wherein the step of preparing the first drug polymer solution further comprises the step of adding a non-solvent to said first polymer solution for a final concentration of about 95% solvent and about 5% non-solvent.

22. The method for preparing a controlled release coating according to claim 15 wherein the step of preparing the first drug polymer solution further comprises the step of adding a non-solvent to said first polymer solution for a final concentration of about 70% solvent and about 30% non-solvent.

23. The method for preparing a controlled release coating according to claim 15 wherein said drug-polymer solution is deposited on the surface of said medical device by a method comprising:
spraying said drug-polymer solution onto said medical device;
drying said medical device overnight at room temperature; and
annealing said medical device at 45° C. for 2 hours.

24. The method for preparing a controlled release coating according to claim 15 wherein said medical device is a stent.

25. The method for preparing a controlled release coating according to claim 24 wherein said stent is a stent graft.

26. The method for preparing a controlled release coating according to claim 15 further comprising a parylene primer coat.

27. The method for preparing a controlled release coating according to either of claims 15 or 26 further comprising a cap coat.

28. The method for preparing a controlled release coating according to claim 15 wherein said drug is an effective amount of an anti-restenotic drug.

29. The medical device of claim 15 wherein said medical device is delivered to the treatment site of a mammal in need thereof.

30. The medical device of claim 29 wherein said medical device is a vascular stent or a stent graft.

31. The medical device of claim 30 wherein said vascular stent or stent graft is delivered to said treatment site using a balloon catheter.

32. A drug-polymer coating for use on a vascular stent or stent graft, the drug-polymer coating comprised of layers of varying porosity.

33. The drug-polymer coating of claim 32 further comprising a first drug polymer layer having a first porosity value made from a first drug polymer solution and a second drug polymer layer having a second porosity value made from a second drug polymer solution.

34. A method for treating restenosis in a mammal in need thereof comprising administering a vascular stent with a polymer coating of gradient porosity for release of an effective amount of an anti-restenotic drug.
